(19) United States
(12) Patent Application Publication
(10) Pub. No.: US 2006/0212100 A1
(43) Pub. Date: Sep. 21, 2006

(54) Non-intrusive laser irradiation device

(75) Inventors: Daniel Jih Huah Wu, Chung Li City (TW); Sheng-Pin Su, Chung Li City (TW)

Correspondence Address: TROXELL LAW OFFICE PLLC, SUITE 1404, 5205 LEESBURG PIKE, FALLS CHURCH, VA, 22041, US

(73) Assignee: EINS OE-TECH CO., LTD.

(21) Appl. No.: 11/079,227

(22) Filed: Mar. 15, 2005

(51) Int. Cl. A61N 5/06

(52) U.S. Cl. 607/93; 607/90; 607/89

(57) ABSTRACT

A non-intrusive laser irradiation device has a shell body, a circuit board, at least two laser components, a switch and a light guiding pipe. The circuit is disposed in the shell body. The laser components are arranged in the shell body. One end of each laser component protrudes out of the shell body, and the other end is electrically connected to the circuit board. One end of the switch is disposed on the shell body, and the other end is electrically connected to the circuit board. The light guiding pipe is sleeved onto the front end of the laser components. Infrared pulse lights with a wavelength of 800-900 nm generated by the laser components are used to cure ache and stimulate the proliferation of marrow cells. The light guiding pipe collects the laser light energy to irradiate directly acupuncture points to achieve the effect of acupuncture and moxibustion.

### BACKGROUND OF THE INVENTION

1. Field of the Invention

The present invention relates to a non-intrusive laser irradiation device and, more particularly, to a laser irradiation device that emits an infrared pulse light with a wavelength of 800-900 nm and an output power of 60 mW and is used for curing degenerate osteoarthritis, gout, bone harm and muscle ache and enhancing the proliferation of marrow cells and joint cartilage.

2. Description of Related Art

The principle of phototherapy was established in late 19^{th} century by the Nobel laureate N. R. Finsen. He used light for curing skin disease. Phototherapy is now used for curing psoriasis, kernicterus and cancer. Development of phototherapy is mainly ascribed to the introduction of laser therapy originally used in surgery. The development of the Infrared (830 nm) AlGaAs semiconductor laser and the red (633 nm) low-power He—Ne laser lead phototherapy to wound healing and pain killing. Many researchers have mentioned the use of the low-power laser therapy for curing various diseases and pains and stimulating growth and regeneration of cells and tissues. For instance, low-power laser therapy can be used for curing degenerate osteoarthritis, gout, bone harm and muscle ache and enhancing the proliferation of marrow cells and joint cartilage. Therefore, low-power laser therapy is an important substitutive therapy for these diseases.

### SUMMARY OF THE INVENTION

An object of the present invention is to provide a non-intrusive laser irradiation device that emits an infrared pulse light with a wavelength of 800-900 nm and an output power of 60 mW and is used for curing degenerate osteoarthritis, gout, bone harm and muscle ache and enhancing the proliferation of marrow cells and joint cartilage.

Another object of the present invention is to provide a non-intrusive laser irradiation device having the function of acupuncture and moxibustion.

To achieve the above objects, the present invention provides a non-intrusive laser irradiation device, which comprises a shell body, a circuit board, at least two laser components, a switch and a light guiding pipe. The circuit is disposed in the shell body. The laser components are arranged in the shell body. One end of each laser component protrudes out of the shell body, and the other end is electrically connected to the circuit board. One end of the switch is disposed on the shell body, and the other end is electrically connected to the circuit board. The light guiding pipe is sleeved onto the front end of the laser components.

### BRIEF DESCRIPTION OF THE DRAWINGS

The various objects and advantages of the present invention will be more readily understood from the following detailed description when read in conjunction with the appended drawing, in which:

FIG. 1 is an exploded perspective view of a non-intrusive laser irradiation device according to a preferred embodiment of the present invention;

FIG. 2 is a perspective assembly view of a non-intrusive laser irradiation device according to the preferred embodiment of the present invention;

FIG. 3 is a cross-sectional view of a non-intrusive laser irradiation device according to the preferred embodiment of the present invention;

FIG. 4 is a rear view of a non-intrusive laser irradiation device according to the preferred embodiment of the present invention; and

FIG. 5 is a perspective view of a non-intrusive laser irradiation device matched with a cover body according to the preferred embodiment of the present invention.

### DETAILED DESCRIPTION OF THE PREFERRED EMBODIMENTS

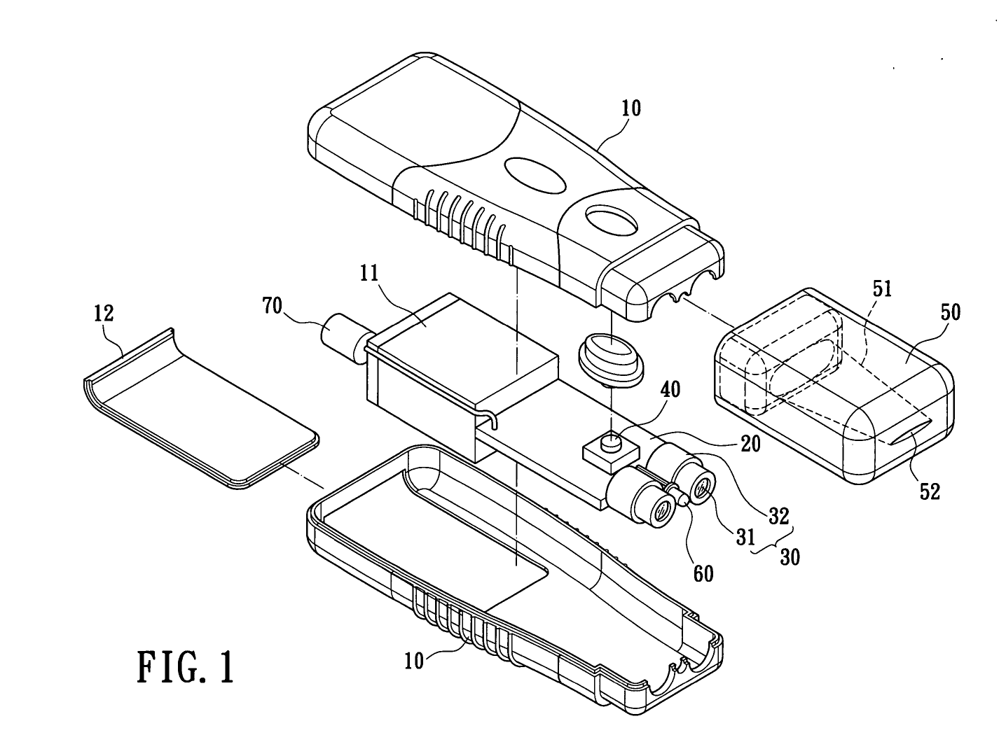

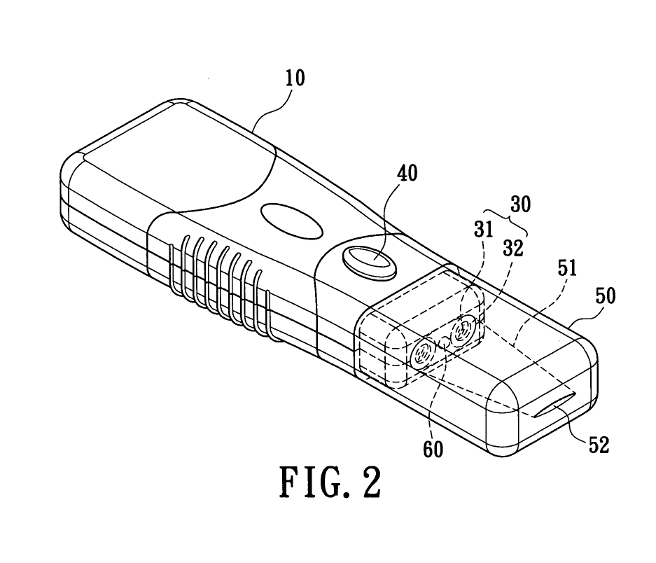

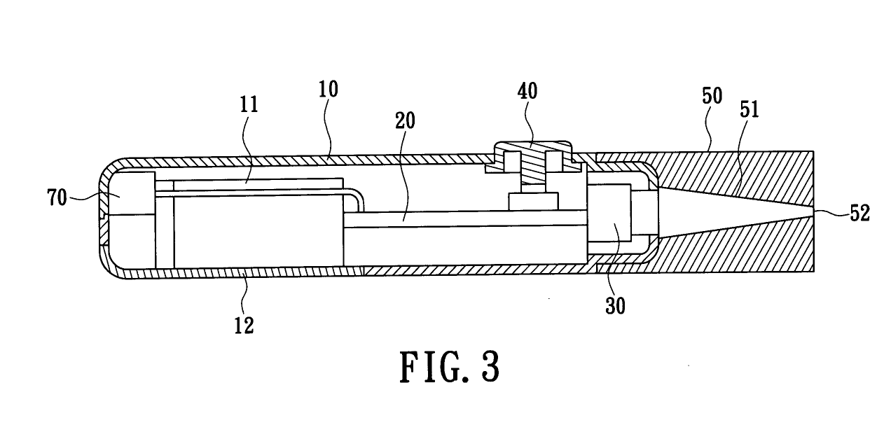

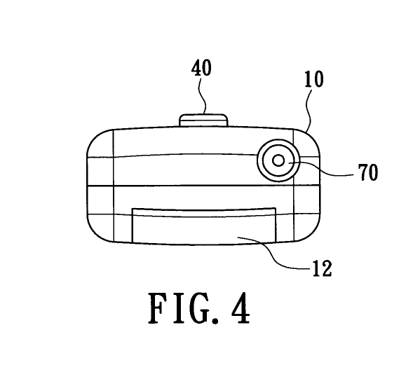

As shown in FIGS. 1 to 4, the present invention provides a non-intrusive laser irradiation device comprising a shell body 10, a circuit board 20, two laser components 30, a switch 40, a light guiding pipe 50, a display lamp 60 and a power connector 70.

The shell body 10 is composed of two, half shell bodies. A battery case 11 for placement of a battery is formed in the shell body 10. A battery case cover 12 is disposed outside of the shell body 10. The battery case cover 12 is combined with the shell body 10 to close the battery case 11.

The circuit board 20 is disposed in the shell body 10. The circuit board 20 is used to control the operation of the laser components 30. The circuit board 20 is connected to the power source via conducting wires. The power source is a DC power source.

The laser components 30 are arranged in the shell body 10. One end of each laser component 30 protrudes out of the shell body 10, and the other end thereof is electrically connected to the circuit board 20. Each laser component 30 has a semiconductor solid-state laser 31 and a metal housing 32. The front and read ends of the metal housing 32 are open. The semiconductor solid-state lasers 31 are received in the metal housings 32. One end of each semiconductor solid-state laser 31 extends to form a solder end soldered onto the circuit board 20 so that the laser component 30 is electrically connected to the circuit board 20. The semiconductor solid-state laser 31 can produce an infrared pulse light with a wavelength of 800-900 nm. The circuit board 20 controls the output power of the laser light to be 60 mW with a repetitive rate of 10 Hz. This lower-power infrared laser pulse light can effectively relieve pains of various muscles, bones and joints, improve blood circulation, eliminate fatigue and stiffness of muscle and bone, relieve neuralgia of muscle and bone and improve gout. This lower-power infrared laser pulse light is suitable for eliminating stiffness of muscle and bone of the old and caused by improper exercise and posture, and can stimulate proliferation of marrow cells and joint cartilage. In other words, the present invention provides a painless, drug-free and non-intrusive laser irradiation device to accomplish the effect of phototherapy.

One end of the switch 40 is disposed on the shell body 10, and the other end thereof is electrically connected to the circuit board 20. The circuit board 20 controls the operation of the laser components through the switch 40.

The light guiding pipe 50 is sleeved onto the front end of the laser components 30. The light guiding pipe 50 has a conical space therein. A light guiding plane 51 is formed on the inner surface of the light guiding pipe 50. After irradiating the light guiding plane 51 and being reflected many times along the axial direction, the laser light is focused into the emissive hole 52 and projected out through the emissive hole 52. The energy of the laser light is thus focused into a point. The light guiding pipe 50 is used to focus the laser light to increase the output intensity of the laser light. The laser light can be used not only to irradiate wounded areas, but also to irradiate directly acupuncture points to achieve the effect of acupuncture and moxibustion. Further, the light guiding pipe 50 can also be removed when using the present invention to cure wounds. The infrared light pulse directly irradiates the wounds to accomplish the effect of phototherapy. The distance between the light beam and the skin should be properly controlled to achieve the optimum effect.

The display lamp 60 is a light-emitting diode electrically connected to the circuit board 20. The display lamp 60 is used to indicate that the laser components 30 are emitting infrared pulse lights. Because infrared light is invisible, after a user presses on the switch 40, the display lamp 60 is turned on to indicate that the laser components are operating.

The power connector 70 is electrically connected to the circuit boards via conducting wires. One end of the power connector 70 protrudes out of the shell body 10. The power connector is connected with a power adaptor for converting an AC power into a DC power provided for the laser components 30.

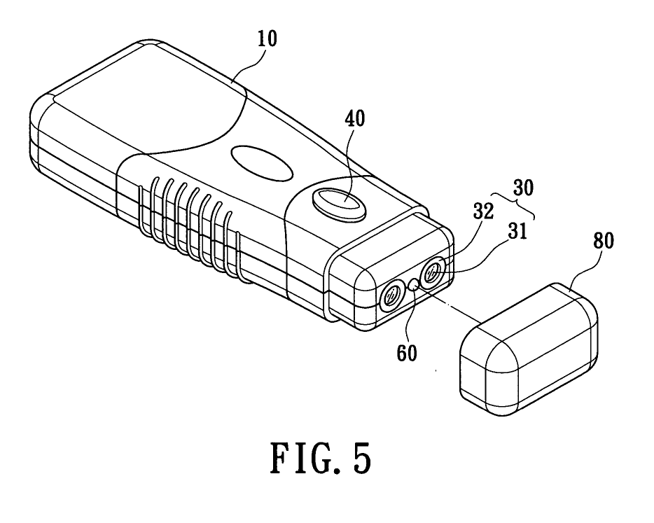

As shown in FIG. 5, the non-intrusive laser irradiation device of the present invention further comprises a cover body 80. The cover body 80 is sleeved onto the front ends of the laser components 30 and used as a protection cover of the laser components 30. When one finishes use of the non-intrusive laser irradiation device, he can remove the light guiding pipe 50 and then sleeve the cover body onto the front end of the laser components 30 to protect them. When one wants to use the non-intrusive laser irradiation device, he can remove the cover body 80 and then sleeve the light guiding pipe 50 onto the laser components 30 or remove the cover body 80 and directly irradiate wounds.

During treatment, the non-intrusive laser irradiation device can directly irradiate pulse light onto wounds. The pulse light treatment lasts about 10 min, and can be performed at least once every day. This phototherapy can reduce the use of other drugs, but cannot replace the prescription of a doctor without permission from the doctor.

To sum up, the non-intrusive laser irradiation device of the present invention has the following characteristics:
1. The present invention makes use of laser components to produce low-power laser pulse light with a wavelength of 800-900 nm. This infrared pulse light can effectively relieve pains of various muscles, bones and joints, improve blood circulation, eliminate fatigue and stiffness of muscle and bone, relax neuralgia of muscle and bone and improve gout. This lower-power infrared laser pulse light is suitable for eliminating stiffness of muscle and bone of the old and caused by improper exercise and posture, and can stimulate proliferation of marrow cells and joint cartilage. In other words, the present invention provides a painless, drug-free and non-intrusive laser irradiation device to accomplish the effect of phototherapy. Because the output power of the laser components 30 is 60 mW, the non-intrusive laser irradiation device is a medical treatment apparatus that can be safely used at home.
2. The present invention makes use of the light guiding plane 51 on the inner surface of the light guiding pipe 50 to focus the laser light and then project the laser light out through the emissive hole 62. The energy of the laser light is thus focused into a point. In other words, the light guiding pipe 50 is used to focus the laser light to increase the output intensity of the laser light. In addition to irradiating wounds, the laser light can also irradiate acupuncture points to achieve the effect of acupuncture and moxibustion. Further, the light guiding pipe 50 can also be removed to irradiate the infrared pulse light directly onto wounds for accomplishing the effect of phototherapy.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A non-intrusive laser irradiation device, comprising:
a shell body;
a circuit board disposed in said shell body;
at least two laser components arranged in said shell body, one end of each of said laser components protruding out of said shell body, and another end thereof being electrically connected to said circuit board;
a switch, one end thereof being disposed on said shell body and another end thereof being electrically connected to said circuit board; and
a light guiding pipe sleeved onto a front end of said laser components.

2. The non-intrusive laser irradiation device as claimed in claim 1, wherein the light emitted by said laser components is an infrared pulse light with a wavelength of about 800-900 nm.

3. The non-intrusive laser irradiation device as claimed in claim 1, wherein a battery case is disposed in said shell body.

4. The non-intrusive laser irradiation device as claimed in claim 1, further comprising a cover body, said cover body being sleeved onto the front end of said laser components.

5. The non-intrusive laser irradiation device as claimed in claim 1, wherein a display lamp is electrically connected to said circuit board and protrudes out of said shell body.

6. The non-intrusive laser irradiation device as claimed in claim 5, wherein said display lamp is a light-emitting diode.

7. The non-intrusive laser irradiation device as claimed in claim 11, wherein a power connector is electrically connected to said circuit board, and one end of said power connector protrudes out of said shell body.

8. A non-intrusive laser irradiation device, comprising:
a shell body;
a circuit board disposed in said shell body;
at least two laser components arranged in said shell body, one end of each of said laser components protruding out of said shell body, and another end thereof being electrically connected to said circuit board; and
a switch, one end thereof being disposed on said shell body and another end thereof being electrically connected to said circuit board.

9. The non-intrusive laser irradiation device as claimed in claim 8, wherein the light emitted by said laser components is an infrared pulse light with a wavelength of about 800-900 nm.

10. The non-intrusive laser irradiation device as claimed in claim 8, wherein a battery case is disposed in said shell body.

11. The non-intrusive laser irradiation device as claimed in claim 8 further comprising a cover body, said cover body being sleeved onto the front end of said laser components.

12. The non-intrusive laser irradiation device as claimed in claim 8, wherein a display lamp is electrically connected to said circuit board and protrudes out of said shell body.

13. The non-intrusive laser irradiation device as claimed in claim 12, wherein said display lamp is a light-emitting diode.

14. The non-intrusive laser irradiation device as claimed in claim 8, wherein a power connector is electrically connected to said circuit board, and one end of said power connector protrudes out of said shell body.
